(19) United States
(12) Patent Application Publication
(10) Pub. No.: US 2012/0059314 A1
(43) Pub. Date: Mar. 8, 2012

(54) ATTACHMENT FOR A STANDARD SYRINGE AND INJECTION DEVICE FOR NEEDLE-FREE INJECTION

(75) Inventor: Peter Eichhorst, Hohen Neuendorf (DE)

(73) Assignee: PRIMOJEX GMBH, Hohen Neuendorf (DE)

(21) Appl. No.: 13/258,757

(22) PCT Filed: Mar. 16, 2010
(86) PCT No.: PCT/EP2010/053403

§ 371 Date: Oct. 19, 2011

(30) Foreign Application Priority Data
Mar. 25, 2009 (DE) 10 2009 001 858.1

(51) Int. Cl. A61M 5/30

(52) U.S. Cl. 604/68

(57) ABSTRACT

The invention relates to an attachment (10) for a standard injection device (70) for needleless injection of a fluid, having a nozzle adapter (12) and a piston unit (40), wherein the nozzle adapter (12) comprises a skin contact surface (16) arranged on the distal end (14) thereof having an outlet opening (18), a first cylinder section (20) connecting proximally to the outlet opening (18) and a second cylinder section (26) connecting to the first cylinder section (20), and the piston unit (40) is mounted in a movable manner in the first cylinder section (20) to form a piston-cylinder unit and the attachment (10) has a fluid line (48) for transporting fluid from the standard injection device (70) to the outlet opening (18).

The present invention relates to an adapter for a standard syringe device for needleless injection of a fluid, as well as to an injection device for needless injection which includes the adapter according to the invention for a standard syringe device.

Injection devices for needleless injection of an active substance into human or animal tissue are capable of generating a special force profile, which realizes the penetration of the skin by forming an injection channel through an initial brief pulse with a steep slope and a high force. After the injection channel is created, a substantially constant force at a lower level dose must follow the brief pulse with the high force in order to supply the complete active substance. Typically, two or more force sources are used to generate the overall force profile, wherein each of the force sources is used to generate a predetermined single force profile and the cooperation of the force sources then forms the desired overall force profile. Such force sources can be manual operating forces, springs and the like. In some conventional embodiments, the forces of the force sources are converted and/or transformed by suitable gears.

A device for injecting a fluid is known from WO 2005/23343 A1, wherein a single-use syringe can be used for needleless injection through use of an adapter. In particular, FIG. 10 of this document illustrates that the force from a spring is used for needleless injection of a dispersion fluid or a fluid to be injected from an actual or from the single-use syringe. The problem associated with this embodiment is that the force of the spring is used both for creating an injection channel and for dispersing the fluid. Furthermore, this device is not capable of reliably generating the desired force profile at an initially very high level and steep slope and with a subsequent lower constant force curve.

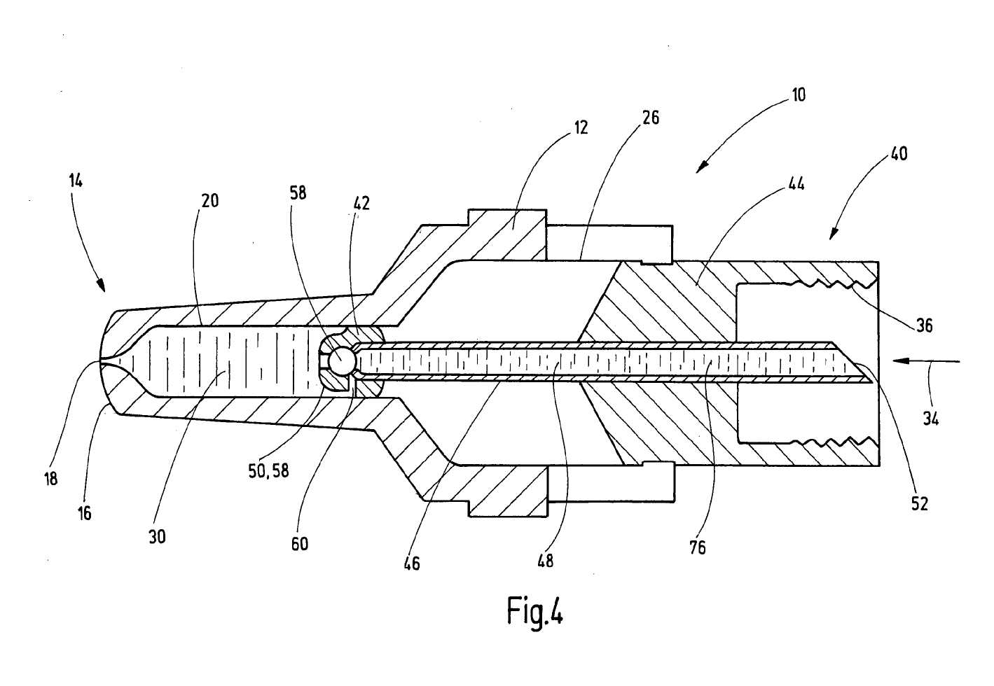

The system illustrated in the document DE 10 2007 008 597 A1 for injecting a fluid through or into the human skin is according to FIG. 4 and the associated description suitable to produce the desired aforedescribed pressure profile. For this purpose, an auto-injector is used which operates an ampoule piston such that the dispersion fluid is first expelled with the desired high force level and subsequently with a smaller force. In this embodiment, the auto-injector must include a force-producing device with which the desired, relatively complicated force profile can be generated. This requires a relatively complex structure and thus relatively high manufacturing and material costs.

DE 10 2006 041 499 A1 shows a particular embodiment of an injection device which includes an operating device adapted to receive a syringe device with a needle such that the active substance stored in the syringe device can be injected needleless. This function is provided by a hollow piston rod of the syringe device which is in fluid connection with the cylinder of the syringe device storing the active substance. The hollow piston rod ends in an outlet opening in a compressed part suitable to be pressed onto the skin for realizing the needleless injection. Two force sources in form of two compression springs are provided for operating this device. Because two force sources are required, the structural complexity and the manufacturing and material costs of the device increase.

WO 2003/105934 A1 discloses a device for needleless injection of a medium into human or animal tissue, which includes a chamber for receiving a pre-injection medium and to be placed on the skin and which is suitable to be connected with a syringe device having a dispersion fluid. A piston receiving the pre-injection medium is arranged in the chamber, wherein the piston together with a cylinder forming the chamber forms a piston-cylinder unit. The force of a compression spring operates on the piston to move the piston and inject the pre-injection medium. With this pre-injection device in cooperation with a syringe device, the pre-injection medium can thus be injected when releasing the spring force, creating the injection channel. By operating the connected syringe device, the dispersion fluid can subsequently be introduced into the tissue through the created injection channel. However, an additional force from an additional force source operating on the syringe device must be provided for introducing the dispersion fluid. In other words, as already described above, two forces or two mutually independently operating force sources are also required in this device for generating the desired force profile, wherein the structure is relatively complicated and the associated manufacturing costs are relatively high.

It is the object of the present invention to provide a device with a simple structure for generating the desired force profile for needless injection and to enable the use of a single force source for expelling a penetration fluid and a dispersion fluid.

The object is solved by providing an adapter for a standard syringe device for needleless injection of a fluid, with a nozzle adapter and a piston unit, wherein the nozzle adapter comprises a skin contact surface arranged at its distal end with an outlet opening, a first cylinder section proximally joined to the outlet opening and a second cylinder section joined to the first cylinder section, and the piston unit is displaceably supported in the first cylinder section for forming a piston-cylinder unit, and the adapter has a fluid line for transporting fluid from the standard syringe device to the outlet opening. According to the invention, the piston unit includes a piston arranged in the first cylinder section and a proximal connection part mechanically connected with the piston, with the proximal end of the connection part (44) designed for mechanical connection of a standard syringe device receiving or capable to receive the fluid, so that a force effect from the standard syringe device on the connection part can be realized for displacing the piston unit and expelling a fluid from or drawing a fluid in from the first cylinder section.

The aforementioned standard syringe device may be a manually fillable syringe for single use, a pen or a pre-filled single-use syringe.

According to the invention, the novel adapter is a device which has a nozzle for expelling and/or receiving fluid.

The diameter of the first and the second cylinder sections may be different; preferably the diameter of the second cylinder section is greater than the diameter of the first cylinder section.

The force effect from the standard syringe device is particularly transmitted to the adapter when the standard syringe device moves. The piston is moved due to the mechanical coupling between adapter and piston, so that a fluid can be expelled from or suctioned into the first cylinder section. The force for moving the standard syringe device can be introduced into its cylinder or piston.

The adapter can be constructed for attaching and/or receiving a Luer fitting of a manually fillable single-use syringe, a plunger of a prefilled single-use syringe or of a cartridge closure of a pen injector.

The fluid line is used for transporting a fluid received in the standard syringe device into the first cylinder section and/or vice versa. The piston has a radial seal relative to the first cylinder section.

The adapter according to the invention with connected standard syringe device enables its initial filling and, in particular in combination with an injection device, the generation of a pressure pulse for penetration. The entire dispersion volume is injected with the needleless injection by operating the piston-cylinder system of the standard syringe device.

In other words, with the adapter according to the invention, the desired pressure peak for penetrating the dermis and hence creation of a penetration channel is initially realized with a small fraction of the injection volume, whereas the remaining injection volume is subsequently injected at a lower pressure (dispersion).

According to the invention, when a pulse-like defined force is applied, which can be manually produced by pressing the injection device onto the injection location or by releasing an internal force source in an injection device, the penetration pressure pulse is generated by having the entire standard syringe device directed operating and displacing the piston of the adapter.

This direct operation can be initiated with an injection device in which the standard syringe device is received. The penetration pressure pulse causes a pulse-like ejection of a fluid received in the first cylinder section from the outlet opening, so that a penetration channel is created in the tissue contacting the outlet opening.

The dispersion pressure is subsequently generated with the same force source by operating the piston of the piston-cylinder system of the standard syringe device. The ratio of the cylinder-surface area of the standard syringe device to the nozzle adapter is dimensioned so that both the penetration and the dispersion pressure can be generated with the desired force profile curves with a single force source. The dispersion fluid is transported from the standard syringe device through the fluid line to the outlet opening, where it can enter the tissue through the created penetration channel.

In other words, according to the invention, the desired pressure profile is generated in a simple manner from the dispersion force source alone by way of the structural design of an adapter according to the invention in conjunction with a standard syringe device.

According to a particular advantage of the invention, the required initial penetration pressure peak is generated—without reaction—by initially displacing the standard syringe system against the nozzle adapter. This prevents loading of the connected standard syringe device by a high pressure pulse.

The adapter according to the invention can be fully used in conjunction with all common primary packing means and disposable syringes as well as with standard syringe devices, such as manually fillable syringes and pre-filled syringes or cartridge-pen systems.

For generating the penetration and the subsequent dispersion pressure profile, the generation of a single common driving force is sufficient, which significantly simplifies the construction of the adapter according to the invention as well as of an injection device for receiving and using the adapter according to the invention.

Advantageously, the piston and the connection part are mechanically connected with each other by a piston rod, wherein the piston rod is constructed hollow for the purpose of fluid transport and thus implements the fluid line between a connected or connectable standard syringe device and the first cylinder section.

In this embodiment, the connection part may also be constructed to be hollow in a partial region and have an outlet opening, wherein the hollow region of the connection part is connected with the hollow piston rod, so that the fluid from the standard syringe device can flow into the first cylinder section via the hollow connection part and the hollow piston rod, from where the fluid can then flow into the tissue through the outlet opening and the channel created with the penetration fluid.

In one embodiment of the invention, the first cylinder section has a first expansion joined to the region of the outlet opening and a second expansion arranged in the proximal region of the first cylinder section, wherein the piston is arranged at the distal end of the hollow piston rod, wherein a sealing element is disposed on the exterior wall of the hollow piston rod, and wherein one or more transverse openings are disposed in the hollow piston rod between the piston and the sealing element, and wherein the piston is designed so that a corresponding gap between the piston and the interior wall of the expansion remains when the piston is positioned in one of the expansions.

In all other positions of the piston in the first cylinder section outside the expansions, the piston radially sealed against the wall of the first cylinder section.

The sealing element also seals against the wall of the cylinder section and thus implements sealing of the space in the first cylinder section on one side, even if the piston is in a position where the fluid can flow around the piston. In other words, during the insertion motion of the piston into the first cylinder section, when the piston is not located in one of the expansions, a double sealing action exist between the hollow piston rod and the first cylinder section, namely by the piston itself and by the sealing element.

This has the advantage that in a situation, when the piston unit is fully retracted and the penetration fluid is expelled, fluid can flow from the standard syringe device through the hollow piston rod, exit from its transverse opening, flow around the piston and exit from the outlet opening.

Conversely, fluid can be suctioned or drawn into a standard syringe device, wherein a vacuum is produced in the first cylinder section when the piston is retracted, e.g., based on a pullback motion of the standard syringe device, whereby fluid is drawn from a reservoir. When the piston reaches an end position and is positioned in the proximal expansion, fluid flows around the piston while the vacuum is maintained, so that the fluid can enter the transverse opening and flow through the hollow piston rod to the connection part and from there into the standard syringe device.

In an alternative embodiment, the piston is arranged at the distal end of the hollow piston rod, which includes an integrated check valve, wherein the blocking effect of the check valve occurs when a fluid flows into the hollow piston rod at the distal end. Advantageously, the check valve is implemented as a ball valve.

For realizing a painless pressure by the adapter on the skin and a sufficient seal of the created or to be created penetration channel, a proximally set-back support shoulder may be joined to the skin contact surface without substantially forming an edge.

In order to be able to connect a pen with integrated cartridge and not only single-use or multiuse syringes as a standard syringe device, the hollow piston rod according to the invention may be proximally guided through the connection part and may have a polished section after the connection part, making the hollow piston rod suitable for piercing a seal of a pen-cartridge. In this embodiment, a thread, preferably an interior thread, is arranged on the connection part for screwing in the pen cartridge.

For attaining the object, an injection device for needless injection with an adapter according to the invention for a standard syringe device and a receiving part, in which a standard syringe device can be received or is received, can also be provided. The injection device furthermore has a handle part for applying a force to the standard syringe device for displacing the standard syringe device to expel a penetration fluid and to operate a standard syringe device piston for expelling a dispersion fluid from the standard syringe device. The receiving part and the handle part are arranged for displacement relative to each other.

Displacement of the standard syringe device causes a displacement of the piston unit of the adapter according to the invention and hence an expulsion of the penetration fluid.

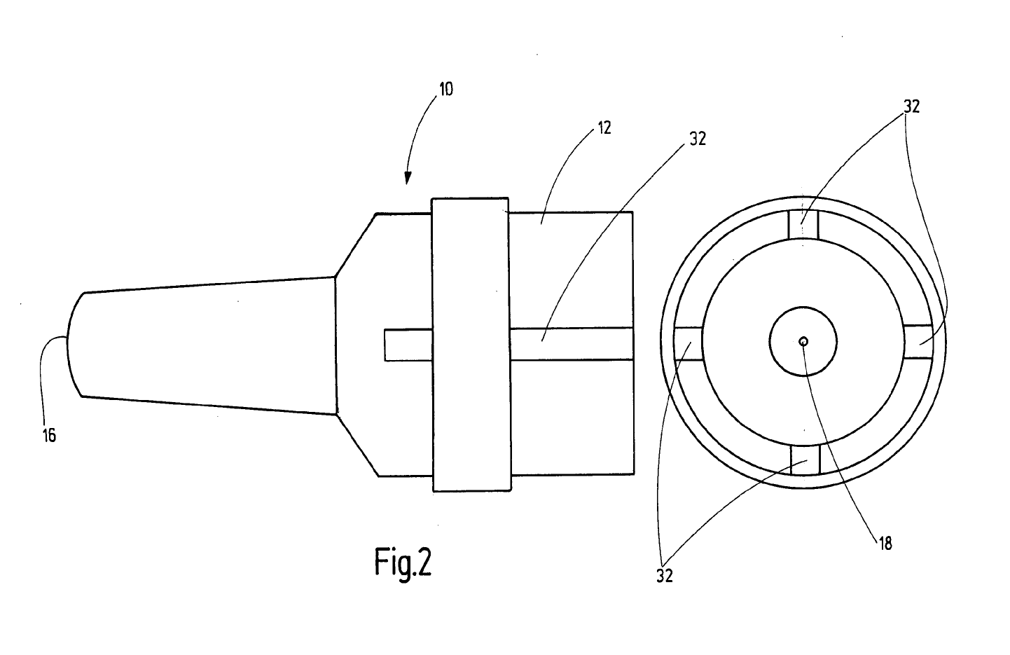

The device for needless injection can also be designed to receive a standard syringe device, as illustrated in FIG. 2 of DE 10 2006 041 499 A1, meaning that the hollow piston rod extends through the piston into the reservoir of the injection device, wherein the connection part in this case is the plug arranged in the interior space.

In this embodiment, too, movement of the standard syringe device causes displacement of the piston unit, so that the penetration fluid is expelled and the plug is subsequently pressed into the interior space of the standard syringe device and hence causing the fluid to be transported from the interior space through the hollow piston rod to the outlet opening.

The injection device according to the invention is hence suitable for the needless subcutaneous or intradermal injection of liquid active substances by using a standard syringe device in combination with an adapter according to the invention, consisting of a nozzle adapter and a piston unit.

The standard syringe device can be a manually fillable single-use syringe, a pre-filled single-use syringe or a pen injector, wherein a force can be applied to the standard syringe device for displacing the piston unit of the adapter according to the invention and expelling the penetration fluid with manual force or also with a constant force source.

The injection device according to the invention has advantages due to the use of the adapter according to the invention which minimizes its complexity and makes freely available the driving force with manual operation.

Advantageously, the injection device has on the handle part a driving device which applies a force to the standard syringe device when the handle part is displaced, thereby causing displacement of the entire standard syringe device.

Moreover, a clamping device can be arranged on the handle part which is used for temporarily securing a standard syringe device piston of a standard syringe device inserted into the injection device.

The clamping device is used to prevent an unintentional displacement of the standard syringe device piston.

In addition, a method for needless injection of a fluid from a standard syringe device into tissue is also provided, which is implemented by using the adapter according to the invention, wherein a single force is used for the injection of a penetration fluid and the subsequent injection of a dispersion fluid.

The method according to the invention can also be performed by using the injection device according to the invention.

The invention will now be explained with reference to the appended drawings.

These show in:

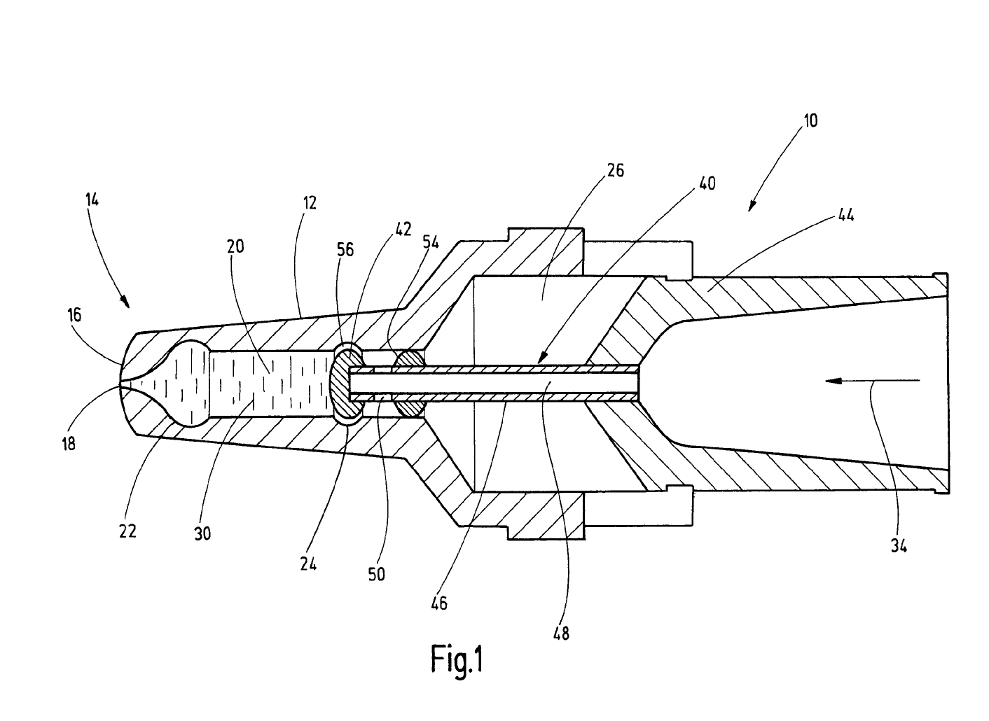

FIG. 1 an adapter according to the invention in a cross-sectional view with a received penetration fluid;

FIG. 2 the adapter according to the invention in a view from the side and from the rear;

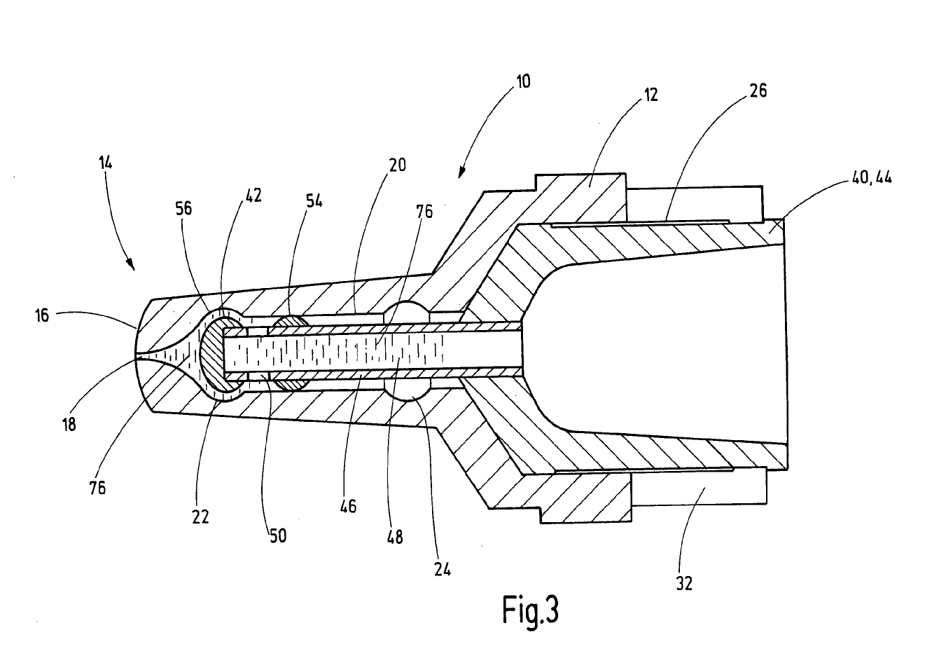

FIG. 3 the adapter according to the invention in a pulled-in cross-sectional view when expelling dispersion fluid;

FIG. 4 an alternative embodiment of the adapter according to the invention in a cross-sectional view; and

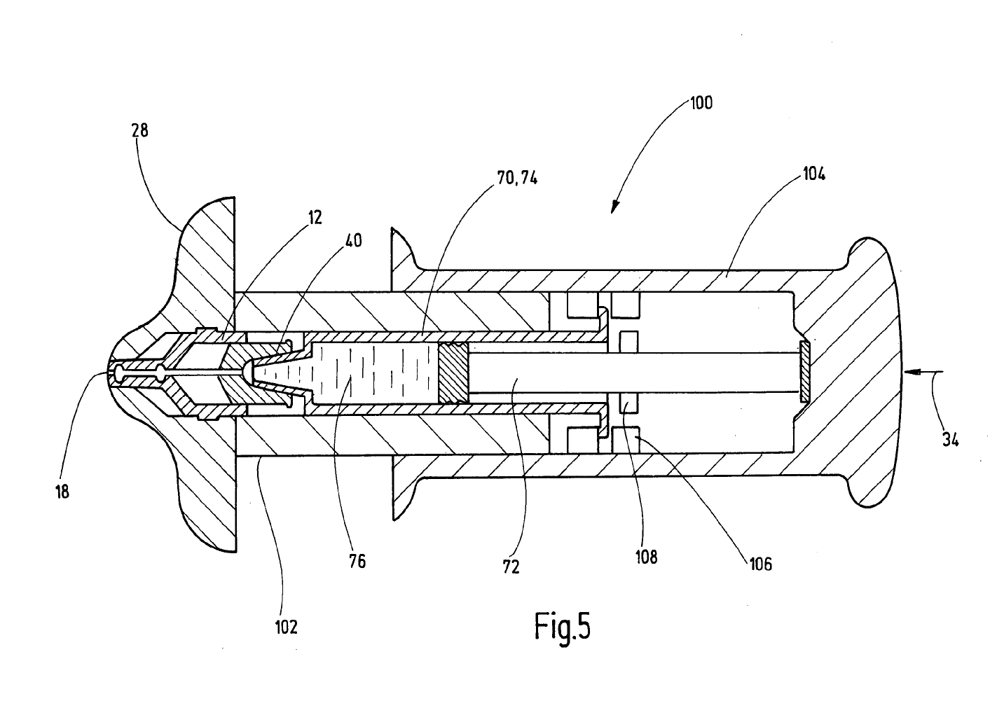

FIG. 5 an injection device according to the invention with a standard syringe device and an adapter according to the invention in a cross-sectional view,

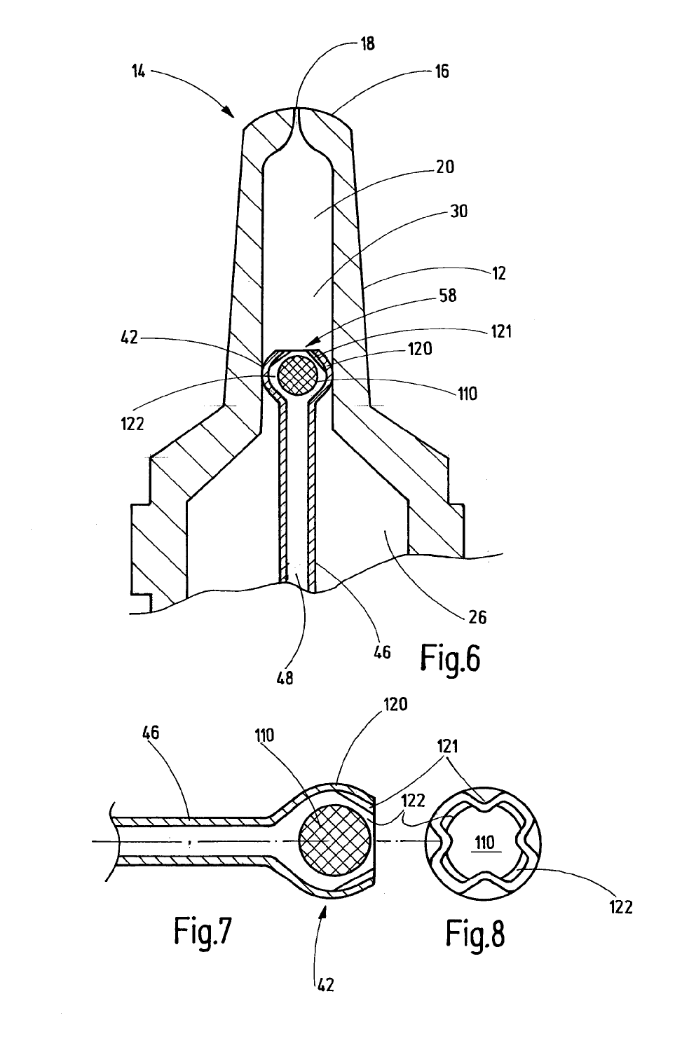

FIG. 6 the adapter according to the invention in a partial cross-sectional view for an alternative embodiment,

FIG. 7 the piston rod with piston in the alternative embodiment according to FIG. 6 in a cross-sectional view, and

FIG. 8 a view of the distal end of the piston according to FIG. 7.

FIG. 1 shows in a cross-sectional view an adapter 10 according to the invention, which includes a nozzle adapter 12 and a piston unit 40, which together form a piston-cylinder unit. The nozzle adapter 12 has at its distal end 14 a skin contact surface 16 for pressing against human or animal skin. An outlet opening 18 for expelling penetration fluid and dispersion fluid is arranged in this skin contact surface 16. The nozzle adapter 12 includes a first cylinder section 20 and a second cylinder section 26 which in the illustrated embodiment each have different diameters. However, the invention is not limited to the cylinder sections with different diameters, but can also be implemented with cylinder sections 20 and 26 having identical diameters. A first expansion 22 is arranged near the distal end 14 in the first cylinder section 20, and a second expansion 24 is arranged proximally. In the illustrated position of the piston unit 40, the penetration fluid 30 is or can also be received in the first cylinder section 20. The piston unit 40 includes a distally arranged piston 42 and a proximal connection part 44 configured to be placed on or attached to a standard syringe device. The piston 42 is connected to the proximal connection part 44 with a hollow piston rod 46. A fluid line 48 is realized by the hollow piston rod 46. One or more transverse openings 50 are arranged in this fluid line 48 and in the hollow piston rod 46, respectively, proximate to the piston 42. A sealing element 54 for sealing the hollow piston rod 46 with respect to the first cylinder section 20 and hence also with respect to the second cylinder section 26 is arranged on the hollow piston rod 46. A gap 56 exists between the piston 42 and the respective expansion 22 or 24 when the piston 42 is located in the region of one of the expansions 22 or 24.

FIG. 1 shows the adapter according to the invention in an initial position in preparation for introducing the penetration fluid into the tissue. A force 34 is applied to the proximal connection part 44 for generating the penetration pressure pulse to expel the penetration fluid 30 from the first cylinder section 20, which causes displacement of the proximal connection part 44, so that the piston 42 is displaced from the second expansion into the cylindrical section of the first cylinder section 20 due to the mechanical coupling between the proximal connection part 44 and the piston 42 via the hollow piston rod 46, thereby expelling from the outlet opening 18 the penetration fluid 30 received in the first cylinder section 20. As can be seen, the pressure surface of the piston 42 and the cross-sectional surface of the first cylinder section 20 are relatively small, so that during application of a force 34 very high pressure is applied to the penetration fluid 30 for realizing the penetration channel in the tissue. The sealing element 44 seals against the hollow piston rod 46 and the first cylinder section 20 during the displacement motion of the piston unit 40. Accordingly, a distal seal is realized by the piston 42 and a proximal seal by the sealing element 44. The force 34 is implemented through the displacement of a standard syringe device joined to the proximal connection part 44, which is not shown in FIG. 1.

FIG. 1 also illustrates the state reached by the adapter of the invention after the adapter is filled. A vacuum is produced in the first cylinder section by a displacement motion of the proximal connection part 44 due to the movement of the connected standard syringe device opposite to the illustrated force 34 into the position illustrated in FIG. 1, which is used for drawing in or receiving the illustrated penetration fluid 30. As can be seen, that with the vacuum in the hollow piston rod 46 due to a vacuum in the standard syringe device connected at the proximal connection part, caused by retraction of the piston from the standard syringe device, fluid can flow around the piston 42 in the first cylinder section 20 through the gap 56 between the piston 42 and the second expansion 24, which then continues to flow through the transverse opening 50 into the fluid line 48 in the hollow piston rod 56 and from there into the standard syringe device received in the proximal connection part.

It is therefore not only possible to inject a penetration fluid 30 through the adapter according to the invention in a simple manner, but to also receive in the adapter according to the invention and in a connected standard syringe device dispersion and penetration fluid from a primary packaging means, for example a vial or a snap-of ampoule. The stroke of the piston unit 40 need only be several millimeters, with advantageous stroke distances being between 5 and 20 mm, in particular between 8 and 12 mm. The penetration fluid volume is advantageously between 20 and 40 μl, in particular 28 to 32 μl.

As seen from FIG. 1, the proximal connection part and thus the standard syringe device rigidly connected with the proximal connection part 44 must be moved for expelling the penetration fluid 30. Accordingly, only a single force needs to be generated for expelling the penetration fluid 30 and the dispersion fluid, because this force 34 is initially used to expel the penetration fluid and is subsequently used to expel the dispersion fluid.

FIG. 2 shows the adapter according to the invention in a view from the front and from the rear. As can be seen, the nozzle adapter 12 has slots 32 which cooperate with complementarily formed form elements on the proximal connection part 44 such that the proximal connection part 44 is formfittingly guided in the nozzle adapter 12. The slots 32 are also used for filling the second cylinder section 26 with air or drawing air from the second cylinder section 26 so as to prevent an overpressure or a vacuum therein.

FIG. 3 shows the adapter according to the invention in a cross-sectional view, where the piston unit 40 and the proximal connection part 44, respectively, are completely retracted into the nozzle adapter 12 or into the first cylinder section 20 and the second cylinder section 26. The penetration fluid 30 illustrated in FIG. 1 has completely been pressed out of the first cylinder section 20 by the displacement of the piston 42. In the illustrated position 42, the piston 42 is located in the first expansion 22, wherein a gap 56 between the piston 42 and the first expansion 22 exists also in this position. Dispersion fluid 76 can now be injected into the previously created penetration channel from an unillustrated standard injection device coupled to the proximal connection part 44 due to an overpressure in the standard syringe device, as illustrated by the hollow piston rod 46, the transverse opening 50, the gap 56 and the outlet opening 18. Return flow of the dispersion fluid 76 into the first cylinder section 20 is prevented by the arrangement of the sealing element 54 which is fixedly arranged on the hollow piston rod 46.

This position of the piston unit 40 illustrated in FIG. 3 also represent the initial position for drawing in and/or filling the standard syringe device and the first cylinder section 20 with the penetration fluid 30. When the proximal connection part 44 moves against the direction of the force illustrated in FIG. 1, the piston 42 is pulled from the first expansion 22 towards the second expansion 24, creating a vacuum in the first cylinder section 20 which causes the first cylinder section 22 be filled with fluid.

In other words, the sealing element 54 closes off the first cylinder section 20. The injectate can now be injected during the dispersion phase of the needleless injection from the exit opening 18 into the tissue through the previously created penetration channel.

FIG. 4 shows an alternative embodiment of the adapter according to the invention in a cross-sectional view, wherein the first cylinder section 20 does not have the expansions shown in FIGS. 1 and 3 and the piston 42 is provided with a check valve 58. This enables expulsion of the penetration fluid 30 from the first cylinder section 20 when the proximal connection part 44 is displaced in the direction of the force 34. After the piston unit 40 has reached its end position in the nozzle adapter 12, the check valve 58 can be opened under increased pressure on the dispersion fluid 46, thereby causing the dispersion fluid to flow out of the outlet opening 18. The dispersion fluid 56 is prevented from flowing out of the outlet opening 18 before the penetration fluid 30 flows out, because a significantly higher pressure is present in the first cylinder section than in a standard syringe device connected to the hollow piston rod 46 when the force 34 is applied and the proximal connection part 44 and the connected piston 42 are displaced. This causes the check valve 58 to close when the proximal connection part 44 is moved in the direction of the force 34.

The adapter according to the invention illustrated in FIG. 4 has the particular feature that a hollow piston rod 20 with a polished section 52 extends through the proximal connection part 44. The proximal connection part 44 can advantageously be screwed onto a distal pen thread representing a fitting for the so-called pen cannulas by way of the thread 36 arranged in the proximal connection part 44, wherein the polished section 52 is used to pierce the hollow piston rod 46 into the pen cartridge of the standard syringe device. In other words, only the cartridge connected to the hollow piston rod is used to fill the first cylinder section 20, so that the first cylinder section is not filled via the outlet opening 18.

The polished section 52 is used for piercing a seal of a connected pen cartridge.

The check valve 58 has a valve channel 60 allowing the dispersion fluid 76 to exit in the flow direction. The piston 42 surrounding the check valve 58 is preferably made of a flexible material which applies an elastically acting force to the ball of the check valve 58 for generating the force causing the check valve effect.

In the presence of overpressure in the hollow piston rod 46, the ball of the check valve 58 is slightly lifted from the closed position of the hollow piston rod 46, allowing dispersion fluid 76 to exit from the hollow piston rod 46 and the valve channel 60.

As can be seen, application of this force creates also in this embodiment initially a very high pressure on the penetration fluid 30, so that the penetration fluid 30 can create the penetration channel in the tissue. When the force 34 is further maintained, it causes the dispersion fluid 76 to exit. The force 34 can hence be realized by operating only one piston of a connected pen cartridge when the piston unit 40 reaches an unillustrated limit stop in the nozzle adapter 12. The injectate from the pen cartridge can now be introduced by opening the check valve 58 through the outlet nozzle 18 into the previously created penetration channel.

If the force 34 is insufficient to produce the required dispersion pressure, then the dispersion phase can also be realized with the device illustrated in FIG. 4 by a consecutive sequence of flooding phases and discharge phases of the first cylinder section 20. The adapter according to the invention operates in cooperation with a connected standard syringe device, for example a pen, in this case cyclically, similar to a pulsating mini-pump, until the preset dose of the dispersion fluid has been dispensed. This means that, when the piston 42 has reached a limit stop at the distal end of the first cylinder section 20, the piston unit 40 can be pulled out again from the first cylinder section 20 in the opposite direction of the force 34, causing the check valve 58 to open and allowing the dispersion fluid 76 to reach the first cylinder section 20. The piston unit is then in a position illustrated in FIG. 4, from which it can be moved again in the direction of the distal end 14 by applying a force 34. The dispersion fluid 76 can hence be pumped into the tissue by successive operation of the piston unit 40 and/or the connected pen.

The embodiment illustrated in FIG. 4 is also suitable for coupling and/or adapting alternative standard syringe devices, such as a pre-filled single-use syringe. Accordingly, the proximal connection part 44 should be designed to be formfitting. The polished cannula section 52 of the extended hollow piston rod 46 is used to pierce through the prefilled single-use syringe according to FIG. 2 of DE 10 2006 041 499 A1. The device disclosed in this document can then be used for applying the force 34.

FIG. 5 shows an injection device 100 according to the invention, with a standard syringe device 70 inserted in its receiving part 102 and a handle part 104 being in contact with its standard syringe device piston 72. An adapter 10 according to the invention with a nozzle adapter 12 and a piston unit 40 is arranged on the standard syringe device 70. The nozzle adapter 12 is joined to a support shoulder 28 for reducing the pressure on the tissue when implementing the needleless injection. The handle part 104 is guided on the receiving part 102. When operating the handle part 104 by applying the force 34, this force 34 is introduced via a driving device 106 into the standard syringe device cylinder 74, causing the standard syringe device 70 to be displaced in the direction of the force and thus pushing the piston unit 70 into the nozzle adapter 12 for ejecting the penetration fluid received in the first cylinder section of the nozzle adapter 12 through the outlet opening 18 in the aforedescribed manner.

A decoupler having a defined force, i.e., a device which no longer applies the force 34 on the standard syringe device cylinder 74 when a predetermined force value has been reached and/or a predetermined travel has occurred, operates in or cooperates with the injection device. The decoupler thus prevents injection of the dispersion fluid 76 before expulsion of penetration fluid 30 when the force 34 is applied on the standard syringe device piston 72.

After coupling, the force is introduced into the standard syringe device piston 72 via the handle part 104, causing the standard syringe device piston 72 to be displaced in the standard syringe device cylinder 74 and the dispersion fluid 76 to be expelled via the hollow piston rod 46. This means that only a single force 34 is required to successively expel the penetration fluid 30 from the first cylinder section 20 and thereafter the dispersion fluid 76 from the standard syringe device cylinder 74.

In an advantageous embodiment, the injection device 100 includes a clamping device 108 which temporarily fixes the standard syringe device piston 72 to prevent an unintentional operation of the standard syringe device 70. This means that according to the invention a needleless injection can be realized with the injection device only by applying a manual force without additional auxiliary energy. By applying a pressure on the skin of about 60 N, a penetration channel can be reliably created through which subsequently the dispersion fluid 76 can be injected. A true penetration pulse for creating the penetration channel can be generated by generating the force 34 and with the different piston surfaces in the first cylinder section 20 and in the standard syringe device 70. When the handle part 104 is displaced further, the penetration channel is used to expel the dispersion fluid 76 during the dispersion phase.

FIG. 6 shows an alternative embodiment of the variant illustrated in FIG. 4. In particular, the embodiment illustrated in FIG. 6 includes a special structure of the check valve 58. The hollow piston rod 46 which may also be constructed as a cannula, as shown in FIG. 4, has at its distal end an expansion 120. A ball 110 is arranged in this expansion 120. Due to its circular cross-section, the expansion 120 also forms the piston 42. The piston 42 and/or the hollow piston rod 46 are preferably fabricated from steel and the nozzle adapter from plastic, so that an adequate sealing effect between the piston 42 and the nozzle adapter 12 can be realized with dynamic friction between these two components. The operation of the thereby produced check valve 58 is substantially identical to the operation of the check valve according to FIG. 4. When the piston 42 moves towards the distal end 14 for pressing the penetration fluid 30 out of the nozzle adapter 12, the check valve 58 creates a blocking effect because the ball 110 moves in front of the distal opening of the hollow piston rod 46. After the penetration fluid 30 is expelled and when a pressure is produced in the standard syringe device 70, as illustrated for example in FIG. 5, for expelling the dispersion fluid 76 through the hollow piston rod 46, the ball 110 is lifted from the distal opening of the hollow piston rod 46, allowing the dispersion fluid 76 to flow out through one or several through-openings 122 created between the ball 110 and the expansion 120.

It will be understood that the check valve 58 not only allows the dispersion fluid 78 to flow out when the piston 42 has already reached a position at the distal end 14, but also when the piston 42 is still located at the proximal end of the first cylinder section and the first cylinder section 20 is to be flooded with penetration fluid 30 via the hollow piston rod 46 and the check valve 58.

The actual structure of the check valve 58 illustrated in FIG. 6 can be seen more clearly in FIGS. 7 and 8. It can be seen that the distal end of the hollow piston rod 46 is deformed for creating the expansion 120 such that the distal end of the hollow piston rod 46 has a larger diameter than the other lengthwise regions. The ball 110 is essentially loose in the region of the expansion 120. To prevent the ball 110 from falling out of the expansion 120, the distal end of the hollow piston rod 46 and of the piston 42 produced therefrom is provided with folds 121 oriented along the longitudinal axis of the hollow piston rod 46. These folds 121 cause a reduction in the diameter of the piston 42 at its distal end, so that the thereby created passage in a plane extending through the longitudinal axis of the hollow piston rod is smaller than the diameter of the ball 110. This is clearly illustrated in FIG. 8 which shows that the piston 42 has altogether four folds 121 distributed along its circumference, with two corresponding pair-wise folds 121 forming a constriction in the passage so that the ball 110 is held inside the expansion 120. The expansion 120 and the folds 121 are dimensioned such that the ball 110 can loosely move in the expansion 120 so as to allow contact with and detachment from the distal end of the hollow piston rod 46. FIG. 8 also shows that the cross-section of the piston 42 is essentially round, so that the expansion 120 itself forms the piston, without requiring additional piston seals.

The expansion 120 with the folds 121 can be easily produced by cold-forming of the hollow piston rod 46. The folds 121 than prevent the ball from falling out when the check valve 58 opens and simultaneously allow, when a corresponding pressure is created in the hollow piston rod 46, the respective fluid to flow out of the hollow piston rod 46 through the through-openings 122 formed between the expansion 120, the folds 121 and the ball 110.

However, the invention is not limited to the embodiment illustrated in FIGS. 6, 7 and 8, and the expansion 120 can also be constructed so that its passageway is greater than the diameter of the ball 110 in all planes extending through the longitudinal axis of the hollow piston rod 46. To prevent the ball 110 from falling out of the expansion 120, a blocking element (not illustrated) oriented in the direction of the longitudinal axis may be arranged at the distal end of the piston 42. This blocking element may be constructed as a single-sided section oriented from one side of the piston 42 in the direction of the longitudinal axis, or it can be constructed as a continuous bridge which extends commensurate with a diameter across the distal end of the piston 42, thereby preventing removal of the ball 110 from the expansion 120. In another alternative embodiment of the distal end of the piston 42, a toothed disk which may be form fittingly connected in a simple manner with the edge of the expansion 120 may be arranged in the expansion 120. Through-openings 122 similar to those illustrated in FIG. 8 are created by the tooth gaps of the toothed disk, through which the fluid can exit from the hollow piston rod 46 through the piston 42.

In another unillustrated alternative embodiment, the hollow piston rod 46 according to FIG. 4 may be constructed as a substantially continuous tube which has a section with a greater interior diameter at its distal end. The ball is also arranged in this section having the enlarged interior diameter. The hollow piston rod 46 has, except for the section with the enlarged interior diameter, an interior diameter which is smaller than the diameter of the ball. This prevents the ball 110 from moving through the hollow piston rod 46, and the ball 110 is instead held in the region having the enlarged interior diameter. For this purpose, the distal end of the hollow piston rod 46 is in a similar manner as already described above provided with an element that prevents removal of the ball 110 from the region having the enlarged interior diameter. Such element may also be a rib arranged on one side and extending in the direction of the longitudinal axis of the hollow piston rod 46, or also a bridge which connects one side of the hollow piston rod 46 with the other side. A toothed disk can also be used, as described above. However, such hollow piston rod 46 constructed with a continuous exterior diameter must be provided on its outside with an additional sealing element, which produces in cooperation with the first cylinder section 20 a sealing effect, preferably through contact along the line or through contact over a small ring surface.

### LIST OF REFERENCE SYMBOLS

10 attachment, piston-cylinder unit
12 nozzle adapter
14 distal end
16 skin contact surface
18 outlet opening
20 first cylinder section
22 first expansion
24 second expansion
26 second cylinder section
28 support shoulder
30 penetration fluid
32 slot
34 force
36 thread
40 piston unit
42 piston
44 proximal connection part
46 hollow piston rod
48 fluid line
50 transverse opening
52 polished section
54 sealing element
56 gap
58 check valve
60 valve channel
70 standard syringe device
72 standard syringe device piston
74 standard syringe device cylinder
76 dispersion fluid
100 injection device
102 receiving part
104 handle part
106 driving device
108 clamping device
110 ball
120 expansion
121 fold
122 through opening

1. Adapter (10) for a standard syringe device (70) for needleless injection of a fluid, with a nozzle adapter (12) and a piston unit (40), wherein the nozzle adapter (12) comprises a skin contact surface (16) arranged at its distal end (14) with an outlet opening (18), a first cylinder section (20) proximally joined to the outlet opening (18) and a second cylinder section (26) joined to the first cylinder section (20), and the piston unit (40) is displaceably supported in the first cylinder section (20) for forming a piston-cylinder unit, and the adapter (10) comprises a fluid line (48) for transporting fluid from the standard syringe device (70) to the outlet opening (18), wherein the piston unit (40) comprises a piston (42) arranged in the first cylinder section (20) and a proximal connection part (44) mechanically connected with the piston (42), with the proximal end of the connection part (44) designed for mechanical connection of a standard syringe device (70) receiving or capable to receive the fluid, so that a force effect from the standard syringe device (70) on the connection part (44) can be realized for displacing the piston unit (40) and expelling a fluid from or drawing a fluid in from the first cylinder section (20).

2. Adapter for a standard syringe device according to claim 1, wherein the piston (42) and the connection part (44) are mechanically connected with each other by a piston rod (46), wherein the piston rod (46) is constructed to be hollow for fluid transport and thereby establishes the fluid line (48) between a connected or connectable standard syringe device (70) and the first cylinder section (20).

3. Adapter for a standard syringe device according to claim 2, wherein the first cylinder section (20) has a first expansion (22) joined to the region of the outlet opening (18) and a second expansion (24) arranged in the proximal region of the first cylinder section (20), that the piston (42) is arranged at the distal end (14) of the hollow piston rod (46), that a sealing element (54) is disposed on the exterior wall of the hollow piston rod (46), and one or more transverse openings (50) are disposed in the hollow piston rod (46) between the piston (42) and the sealing element (54), and the piston (42) is designed so that a corresponding gap (56) between the piston (42) and the interior wall of the expansion remains when the piston (42) is positioned in one of the expansions (22, 24).

4. Adapter for a standard syringe device according to claim 2, wherein the piston (42) is arranged at the distal end (14) of the hollow piston rod (46) and comprises an integrated check valve (58), wherein the blocking effect of the check valve (58) occurs when a fluid flows into the hollow piston rod (46) at the distal end (14).

5. Adapter for a standard syringe device according to claim 1, wherein a proximally set-back support shoulder (28) is joined to the skin contact surface (16) without substantially forming an edge.

6. Adapter for a standard syringe device according to claim 1, wherein the hollow piston rod (46) is proximally guided through the connection part and has a polished section proximally behind the connection part (44), making the hollow piston rod (46) suitable for piercing a seal of a pen-cartridge.

7. Injection device for needleless injection, with an adapter for a standard syringe device according to claim 1, and a receiving part, in which a standard syringe device (70) can be or is received, and a handle part (104) for applying a force to the standard syringe device (70) for displacing the standard syringe device (70) to expel a penetration fluid (30) and to operate a standard syringe device piston (72) for expelling a dispersion fluid (76) from the standard syringe device (70).

8. Injection device for needleless injection according to claim 7, wherein a driving device (106) is arranged on the handle part (104), with the driving device (106) causing application of a force on the standard syringe device (70) when the handle part (104) is displaced and hence a displacement of the entire standard syringe device (70).

9. Injection device for needleless injection according to claim 7, wherein a clamping device (108) is arranged on the handle part (104), wherein the clamping device (108) is used to temporarily secure a standard syringe device piston (72) of a standard syringe device (70) placed in the receiving part (102).

10. Method for needleless injection of a fluid from a standard syringe device into tissue, by using the adapter according to claim 1, wherein a single force is used for the injection of a penetration fluid (30) and the subsequent injection of a dispersion fluid (76).
